(19) United States
(12) Patent Application Publication
(10) Pub. No.: US 2020/0254442 A1
(43) Pub. Date: Aug. 13, 2020

(54) BIOLOGICAL SAMPLE KIT

(71) Applicant: Gentueri Inc., Madison, WI (US)

(72) Inventor: Randolph James Nagy, Madison, WI (US)

(21) Appl. No.: 16/788,044

(22) Filed: Feb. 11, 2020

Related U.S. Application Data
(60) Provisional application No. 62/804,409, filed on Feb. 12, 2019.

(51) Int. Cl. B01L 3/00; A61B 10/00

(52) U.S. Cl. CPC B01L 3/508; A61B 10/0096; B01L 2300/021; B01L 2200/0689; B01L 2300/16; B01L 2200/141; B01L 2300/0861

(57) ABSTRACT

A sample kit includes a sealable container having a first region, a second region, and a third region. The first region is arranged to house at least one collection device. The second region is arranged to house at least one sample identification holders. The third region is configured to house a desiccant. The collection device is configured to obtain a sample, where the sample is configured to be positioned within the sample identification holder to define a completed sample. The sealable container includes a coating on at least one of an inner surface of the sealable container or an outer surface of the sealable container.

### CROSS-REFERENCE TO RELATED APPLICATIONS

The present application is related to and claims the priority benefit of U.S. Provisional Patent Application No. 62/804,409, filed Feb. 12, 2019, the entire contents of which are incorporated herein by reference.

### TECHNICAL FIELD

The present disclosure relates to biological sample collection kits, methods of manufacture, and methods of use. In particular, the disclosure relates to sample kits including a sealable container.

### INTRODUCTION

Current sexual assault kits may include swabs for collecting samples and flat swab boxes for storing the samples. The samples that may be collected include oral, vaginal, and/or anal swab samples. The samples are pre-dried before the samples are stored within swab boxes. Typically, the samples are air dried immediately for about one hour to reduce the possibility that moisture in the swabs allow microorganisms to grow, which can destroy the evidentiary value of the swabs. Pre-drying requires the user to manipulate the samples and exposes the samples to possible contamination and misidentification. For example, the user may arrange the samples in a pre-labeled location on a drying rack to identify the samples. The samples may touch each other or be mixed up because each sample is not individually labeled. Once the samples are dried, the user must label and form the swab boxes to allow for the sample to be inserted within the swab boxes. The multiple stages of the collection process provide multiple opportunities for mislabeling and contamination.

### SUMMARY

In one aspect, a sample kit is disclosed. An example sample kit includes a sealable container including a coating on at least one of an inner surface or an outer surface of the sealable container. The sealable container includes a first region arranged to house at least one collection device, and the at least one collection device is configured to obtain a sample. A second region of the sealable container is arranged to house at least one sample identification holder, and the sample is configured to be positioned within the at least one sample identification holder to define a completed sample. A third region of the sealable container is configured to house a desiccant, and the desiccant is configured to be selectively activated to dry completed sample.

In another aspect, an example method of assembling a sample kit is disclosed. The method includes providing a sealable container defining a first region, a second region, and a third region; arranging at least one collection device in the first region; arranging at least one sample identification device in the second region; and providing at least one desiccant in the third region.

Other aspects of the disclosure will become apparent by consideration of the detailed description and accompanying drawings.

### BRIEF DESCRIPTION OF THE DRAWINGS

FIG. 1 is a schematic diagram of an example sampling environment.

FIG. 2 is a top view of an embodiment of a sample kit.

FIG. 3 is a top view of the sample kit of FIG. 2 including at least one collection device, at least one sample identification holder, and a desiccant.

FIG. 4 is a flow chart of an example method of assembling a sample kit.

### DETAILED DESCRIPTION

Generally, the instant disclosure is directed to a sample kit usable for collecting and preserving multiple samples (e.g., secretion samples such as sperm, saliva, blood, etc.), typically at room temperature. Exemplary sample kits can be configured to house a plurality of individual components to collect and preserve the samples. During sample collection, the individual components may require limited assembly and manipulation to collect and preserve the samples. For example, the sample kit may be configured to house and desiccate the samples within the sample kit. Because the sample kit can dry samples therein, instantly disclosed sample kits eliminate the pre-drying step or the need to form and use individual boxes for each sample performed during typical sample collection to ensure samples are dry. As a result, use of disclosed and contemplated sample kits can reduce the number of steps required to preserve the sample, the possibility of contamination, and the possibility of mislabeling the sample.

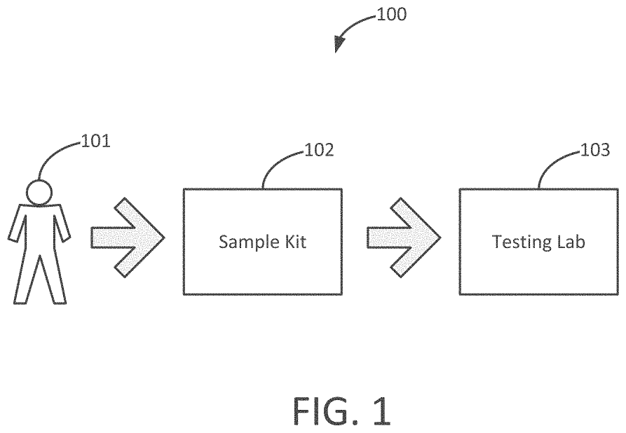

FIG. 1 is a schematic diagram of sampling environment 100. Sampling environment 100 includes a user 101, a sample kit 102, and a testing lab 103. The sampling environment 100 may be part of a method of use, described in greater detail below. The sampling environment 100 may be in various types of locations. In some embodiments, the sampling environment 100 may begin at a crime scene, a hospital, other health facilities, etc. The user 101 may access the sample kit 102, perform the required testing, and the sample kit 102 may be transported to the testing lab 103. In other embodiments, the sampling environment 100 may begin and end with the user 101 and the sample kit 102 in the testing lab 103.

The user 101 may access a sample kit 102. The user 101 may be a person that desires samples to be taken for a variety of tests. In some embodiments, the user 101 may be a victim of sexual assault. A lab technician, a doctor, a nurse, a certified sexual assault health professional, etc. may accompany the user 101 to perform the required tests. The user 101 may access the sample kit 102 in the testing lab 103. Among other embodiments, the user 101 may receive the sample kit 102 in a protected package at a location such as a crime scene.

The sample kit 102 may receive the required samples from the user 101 and desiccate the samples within the sample kit 102. The samples can include secretion samples (e.g. sperm, saliva) collected from areas on the victims related to the crime. The ability to desiccate the sample allows the user 101 to efficiently store and label the samples without the additional steps such as pre-drying the samples or forming individual boxes to ship each sample. The sample kit 102 is configured to continue to desiccate the samples and protect the sample from contamination, spilling, etc.

The sample kit 102 is received at the testing lab 103. The samples provided by the user 101 may be tested at the testing lab 103. The testing lab 103 may be a doctor's office, crime laboratory, an emergency room, a certified DNA testing lab, etc. In some embodiments, the testing lab 103 analyze samples from a sexual assault test such as secretion samples (e.g. sperm, salvia, lubricant, blood). In other embodiments, the samples provided by the user 101 may be a skin, fibers, urine, etc. In other embodiments, a sample provided by user 101 may be used for toxicology, identification, screening or other for other health or medical purposes.

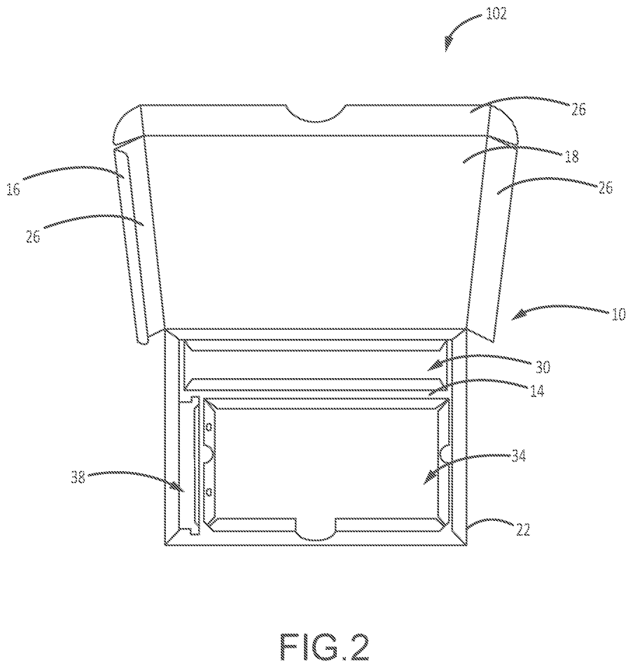

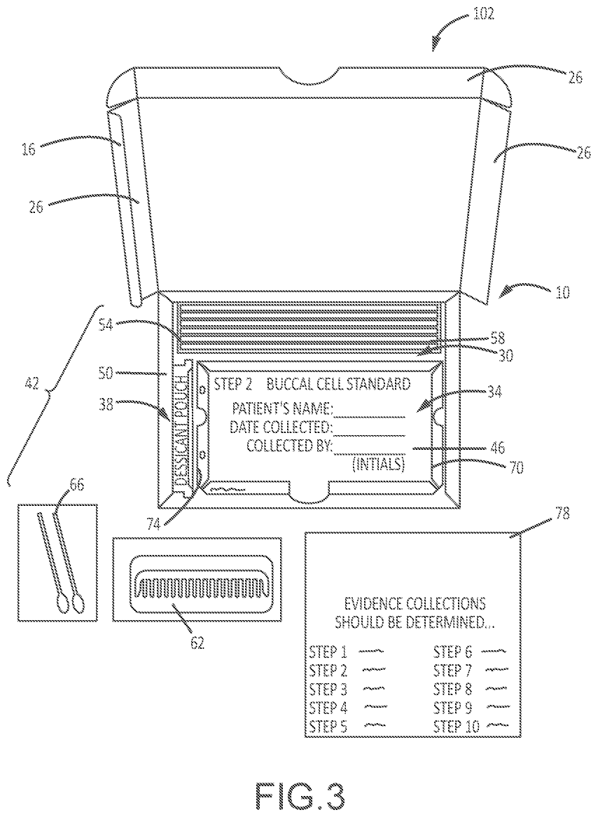

FIG. 2 illustrates an exemplary sample kit 102 in an empty state. The sample kit 102 includes a sealable container 10 having an inner surface 14 and an outer surface 16. A portion of the outer surface 16 is visible in FIG. 2 and FIG. 3 and is defined as an exterior portion of the sealable container 10. The outer surface 16 is exposed to the outside environment when the sealable container 10 is closed (closed orientation not shown in the Figures). The inner surface 14 is defined as any interior surface of the sealable container 10. The inner surface is not exposed to the outside environment when the sealable container 10 is closed.

A moisture barrier coating may be positioned on at least one of the inner surface 14 and the outer surface 16 of the sealable container. The moisture barrier coating of the sealable container 10 is configured to provide protection to the samples positioned inside the sealable container 10 by maintaining a moisture or humidity barrier between the inside of the sealable container 10 and the outside environment. The moisture barrier allows for the samples inside of the sealable container 10 to actively dry, which will be discussed in more detail below.

In some embodiments, the moisture barrier coating may be a polymer or film laminate, which is applied and/or bonded to the outer surface 16 or inner surface 14 of the sealable container 10. The moisture barrier coating may be a coating 0.5 to 3.0 millimeters thick; less than 0.5 millimeters thick; 1.0 mm to 2.5 mm thick; or 1.0 mm to 1.5 mm thick. In some instances, the moisture barrier coating can be 1.0 mm thick; 1.1 mm thick; 1.2 mm thick; 1.3 mm thick; 1.4 mm thick; 1.5 mm thick; 1.75 mm thick; or 2.0 mm thick. In other embodiments, the moisture barrier coating may vary in thickness and be formed with other materials that provide the moisture barrier. The sealable container 10 may also include a tamper-evident seal (not shown) to maintain the integrity of sample kit 102 during transportation.

In the illustrated embodiment, the sealable container 10 includes a lid portion 18 and a base portion 22. The lid portion 18 includes a plurality of flaps 26 that engage with the base portion 22 to seal the sealable container 10. The base portion 22 includes a first region 30, a second region 34 and a third region 38. In the illustrated embodiment, the first region 30, second region 34, and third region 34 are formed as an integral part of the base portion 22. In other embodiments, one or more inserts may be placed within the base portion 22 to define the first region 30, second region 34, and third region 38. In other embodiments, the sealable container 10 may include additional regions to house additional collection or preserving devices or any other equipment required for sampling or region specifically designed to house the sample. The regions of the sealable container 10 may be positioned in any position to allow the regions to house the desired components within the regions.

FIG. 3 illustrates the sample kit 102 of FIG. 2 including the sealable container 10 in a filled state. The sealable container 10 includes the at least one collection device 42, which includes a collection end 54 for obtaining a sample. The collection devices 42 are arranged to be positioned within the first region 30 or the second region 34. In the illustrated embodiment, the collection devices 42 include swabs 58 positioned within the first region 30. Each individual swab 58 may be further positioned in a swab pouch to ensure the integrity of the swabs 58.

The sample kit 102 may also include additional items used for collection other than swabs 58 such as combs 62, specialty swabs 66, specimen cards, or specialty devices. For example, the combs 62 may be configured to collect pubic hairs and the specialty swabs 66 may be configured to collect fingernail evidence. The combs 62, the specialty swabs 66, and other additional items are configured to be positioned on top or within the first region 30 or the second region 34 (not shown). In other embodiments, the collection devices 42 may be positioned in any order to efficiently house the collection devices 42. Additionally, the first region 30 may be used to store other evidence related to the reason the collection is occurring. For example, after collection, the swabs 58 may no longer be positioned within the first region 30. Therefore, evidence such as undergarments or other relevant evidence may be stored in the first region 30.

The sealable container 10 may be configured to house at least one sample identification holder 46 arranged within the second region 34. The sample identification holders 46 may be breathable to allow air to flow through the sample identification holders. In the illustrated embodiment, the sample identification holders 46 are envelopes. In other embodiments, the sample identification holders 46 may be storage tubes, specimen cards, sterile containers, etc. for storing the sample. The sample identification holders 46 may further include information such as a patient's name, a date collected, and a collector's name or initials. The sample identification holders 46 may be pre-labeled envelopes including the information or the user and/or collector may label the envelopes during the collection process. The sample identification holders 46 may also include an insertion end 70 (e.g., a flap on the envelope) to allow the collection device 42 to be inserted consistently into each sample identification holder 46. The collection device 42 may be inserted with the collection end 54 positioned at an end 74 of the sample identification holder 46 opposing of the insertion end 70.

The term “completed samples” is defined herein as the sample identification holder 46 containing the sample. The sample is configured to be positioned within the sample identification holder 46 through the insertion end 70. Once the sample is housed within the sample identification holder 46, defining the completed sample, the completed sample may be sealed to preserve and protect the sample. In the illustrated embodiment, the completed sample is configured to be arranged within the second region 34. In other embodiments, the sealable container 10 may include a fourth region to house the completed samples.

The sealable container 10 may be configured to house a foil pouch or desiccant pouch 50 arranged within the third region 38. The desiccant pouch 50 contains a desiccant (not shown). The desiccant pouch 50 is configured to be removed from the third region 38 and opened to activate the desiccant. The desiccant is configured to activate in response to the opening of the desiccant pouch 50. The desiccant then actively dries the completed sample positioned within the sealable container 10.

In the illustrated embodiment, the third region 38 is positioned adjacent to the second region 34, which positions the desiccant adjacent the completed sample. The collection end of the collection device 42 (positioned in sample identification holder 46) can be oriented adjacent or, alternatively, opposite the third region 38 (such that the “front of the sample identification holder 46 is facing downward relative to the user opening sealable container 10). The positioning of the third region 38 allows for the desiccant to actively dry the completed sample through the breathable sample identification holder 46 without the need of pre-drying the completed sample before inserting the completed sample within the sealable container 10. In other embodiments, the third region 38 and the desiccant pouch 50 may be positioned within the sealed container. Additionally, there may be multiple desiccants positioned within the sealable container 10 in multiple regions or in a single region.

The sample kit 102 may include additional material such as bags for other materials or evidence and instructions 78. The additional material may be positioned on top of the filled first region 30, second region 34, or third region 38 to ensure the additional material are clearly visible when the sample kit 102 is opened. Additionally, the sample kit 102 may include barcodes and labels to identify the collected samples. The barcodes and labels may be positioned on the bottom of the sealable container 10 (e.g., positioned within any region) to allow the user to apply the labels and barcodes during or after collection.

During an example operation, a user 101 breaks the tamper-evident seals to open the kit. The additional material positioned on the top of the filled first region 30, second region 34, and third region 38 is removed. The additional material may include instructions 78 that the user 101 can read and follow.

A collection device 42 may be removed from the first region 30. The collection device 42 may be in a pouch, which the user 101 opens to remove the collection device 42 for the collection of the sample. A sample is obtained using the collection device 42. After collection of the sample, a sample identification holder 46 such as an envelope is chosen. In some cases, the sample identification holders 46 may be pre-labeled envelopes including the name of whose sample was taken. In other embodiments, the user 101 may label the sample identification holders 46 during the collection process.

Once the sample identification holder 46 is chosen, the collection device 42 may be returned into its original pouch. The user 101 may open the insertion end 70 of the sample identification holder 46 and insert the collection device pouch with the enclosed sample into the sample identification holder 46 with the correct identifying name. Additionally, the collection end 54 of the collection device 42 is positioned at the opposing end 74 of the insertion end 70. The sample identification holder 46 may be positioned within the second region 34 for storage of the completed sample.

The collector repeats the steps outlined above with each of the collection devices 42 in the sample kit 102. Once each of the sample identification devices contains samples, the completed samples are positioned within the second region 34 of the sealable container 10 and they can be in the same orientation the sample identification holders 46 were originally positioned.

In some instances, the collection end 54 of the collection device 42 may be positioned within the sample identification holders 46 adjacent to the third region 38, which includes the desiccant pouch 50 with the desiccant. In other instances, the collection end 54 of the collection device 42 may be positioned opposite the third region 38. The user 101 removes and opens the desiccant pouch 50 to activate the desiccant. The desiccant is removed from the desiccant pouch 50 and positioned within the third region 38. The lid portion 18 of the sealable container 10 is secured to the base portion 22 to secure the completed samples in the sample kit 102.

The design of the sample kit can minimize the distance and obstructions between the collection end 54 of the collection device 42 and the desiccant. The desiccant is configured to dry the completed samples before the samples can start to degrade from microbial activity. Additionally, the coating on the sealable container 10 also minimizes the amount of air drawn into the kit. Once the desiccation is completed, the coating of the sealable container 10 is configured to allow the samples to stay dry.

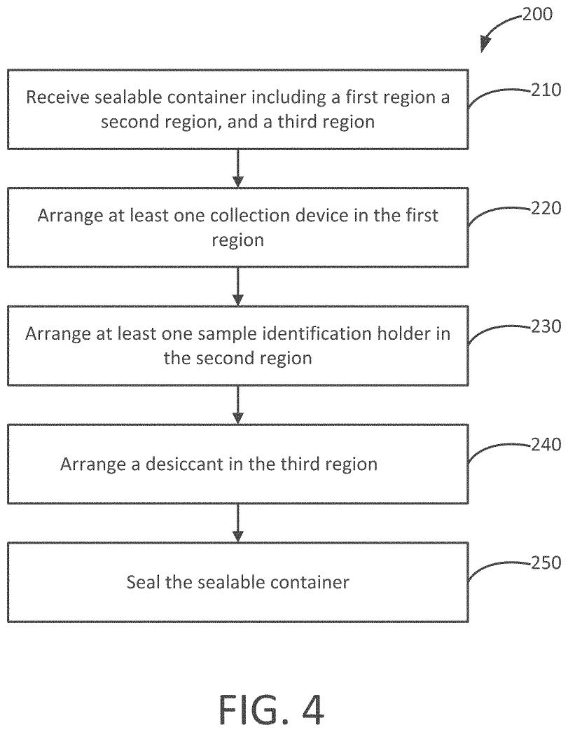

FIG. 4 shows an example method 200 of assembling the sample kit 102 disclosed above. During the method of assembling the sample kit 102, components may be received from an external source (e.g. a manufacturer of desiccants, collection devices, and storage devices) or the components may be manufactured by the assembler.

Method 200 begins by receiving a sealable container 10 (operation 210). The sealable container 10 includes a first region 30, a second region 34, and a third region 38. The sealable container 10 may include the first, second, and third region 38 as an integral part of the sealable container 10. In some embodiments, at least one insert may be arranged within the sealable container 10 to define the first region 30, the second region 34, and the third region 38.

The at least one collection device 42 is positioned within the first region 30 (operation 220). The at least one sample identification holder 46 is positioned within the second region 34 (operation 230). The at least one desiccant is positioned within the third region 38 (operation 240). The at least one collection device 42, the at least one sample identification holder 46, and the at least one desiccant may be positioned within the sealable container 10 in any order (e.g., operations 220, 230, and 240 are interchangeable).

Once the at least one collection device 42, at least one sample identification holder 46, and at least one desiccant within are positioned within the sealable container 10, the sealable container 10 is sealed (operation 250). In some embodiments, the sealable container 10 may be sealed with a tamper-evident seal. Additionally, before the sealable container 10 is sealed, additional material such as instructions 78, other sampling equipment, and/or other information forms may be placed on the top of the first, second and third region 38.

In some embodiments, a moisture barrier coating may be applied to at least one of an outer surface 16 and an inner surface 14 of the sealable container 10. The moisture barrier coating may be applied to the before operation 210 or at any point during the example method 200 of assembling the sample kit 102.

While certain embodiments of the disclosure are explained in detail above, it is to be understood that the disclosure is not limited in its application to the details of construction and the arrangement of components set forth in the following description or illustrated in the following drawings. The disclosure is capable of other embodiments and of being practiced or of being carried out in various ways

What is claimed is:

1. A sample kit comprising:
a sealable container including a coating on at least one of an inner surface of the sealable container or an outer surface of the sealable container, the sealable container comprising:
a first region arranged to house at least one collection device, the at least one collection device configured to obtain a sample;
a second region arranged to house at least one sample identification holders, the sample configured to be positioned within the at least one sample identification holder to define a completed sample; and
a third region arranged to house a desiccant, the desiccant configured to be selectively activated to dry the completed sample.

2. The sample kit of claim 1, wherein the sealable container includes an insert defining the first region, the second region, and the third region.

3. The sample kit of claim 1, wherein the desiccant is positioned within a desiccant pouch positioned in the third region, wherein the desiccant pouch can be removed from the third region and opened to selectively activate the desiccant pouch.

4. The sample kit of claim 3, wherein third region containing the desiccant is configured to be positioned adjacent the second region.

5. The sample kit of claim 4, wherein the completed sample includes a collection end where the sample was obtained, wherein the collection end is configured to be positioned adjacent the desiccant.

6. The sample kit of claim 1, wherein the sealable container includes a tamper-evident seal.

7. The sample kit of claim 1, wherein the coating is configured to minimize the amount of air and moisture drawn into the sample kit.

8. The sample kit of claim 1, wherein the completed sample is received by second region.

9. A method of assembling a sample kit, the method comprising;
providing a sealable container defining a first region, a second region, and a third region;
arranging at least one collection device in the first region;
arranging at least one sample identification device in the second region;
arranging at least one desiccant in the third region; and
sealing the sealable container.

10. The method of claim 9 further comprising sealing the sealable container with a tamper evident seal.

11. The method of claim 9 further comprising arranging additional material such as instructions on top of the first, second, and third regions.

12. The method of claim 9 further comprising arranging a plurality of swabs into the first region defined as the at least one collection device.

13. The method of claim 12 further comprising arranging a comb and specialty swabs on the top of the plurality of swabs or positioned on top or between the at least one sample identification holder.
